SEQUENCE LISTING (3 sequences)

SEQ ID NO: 1 - protein, 15 aa
SQDYENQTWEEYTRT

SEQ ID NO: 2 - protein, 53 aa
MRVASSLFLPVLLTEVWLVTSFNLSSHSPEASVHLESQDYENQTWEEYTRTDP

SEQ ID NO: 3 - protein, 70 aa
NLQVSRVLQQSVLEGGMKRGTEAVSGTLSEIIAYSMPPAIKVNRPFHFIIYEEMSRMLLFLGRVVNPTVL

(19) United States
(12) Patent Application Publication
(10) Pub. No.: US 2002/0142986 A1
(43) Pub. Date: Oct. 3, 2002

(54) Nucleic acids encoding novel serine protease inhibitor proteins associated with the liver and methods of making and using them

(75) Inventor: Anthony F. Purchio, Cardiff, CA (US)

Correspondence Address: GREGORY P. EINHORN, Fish & Richardson P.C., Suite 500, 4350 La Jolla Village Drive, San Diego, CA, 92122, US

(73) Assignee: Hepatix, Inc., a California corporation

(21) Appl. No.: 10/062,023

(22) Filed: Jan. 31, 2002

Related U.S. Application Data
(63) Continuation of application No. 09/038,714, filed on Feb. 3, 1998.
(60) Provisional application No. 60/036,842, filed on Feb. 3, 1997.

(51) Int. Cl. A61K 48/00; A61K 38/17; C07H 21/04; C12P 21/02; C12N 5/06; C12N 9/99

(52) U.S. Cl. 514/44; 514/12; 435/69.2; 435/184; 435/320.1; 435/325; 536/23.2

(57) ABSTRACT

The invention provides human regeneration-associated serpin-1 (RASP-1) polypeptide and nucleic acid molecules that encode RASP-1. Also included in the invention are diagnostic and therapeutic methods using RASP-1 polypeptides and nucleic acids.

### CROSS REFERENCE TO RELATED APPLICATIONS

This application claims priority from Provisional Application Serial No. 60/036,842, filed Feb. 3, 1997, which is incorporated herein by reference in its entirety including all figures and drawings and to which priority is claimed under 35 U.S.C. §119(e).

### BACKGROUND OF THE INVENTION

1. Field of the Invention

The invention relates generally to a family of serine protease inhibitors and specifically to a novel serine protease inhibitor, RASP-1, which is associated with hepatic regeneration, and nucleic acids encoding RASP-1.

2. Description of Related Art

Hepatocellular diseases refer to any number of conditions involving necrosis of hepatic cells due to causes including viral, alcohol, lesions (e.g., tumor) and injury. Treatment of these disorders includes interferon therapy, conventional anti-virus therapy, surgery and the use of various medicines which are generally used in the treatment of hepatitis, for example.

No single classification of the various types of liver disease is entirely satisfactory because in many instances the etiology and pathogenetic mechanism are obscure. As a consequence, one finds an abundance of labels and names applied to hepatic disorders. Some individuals use the term hepatitis to imply viral infection; others use the term to connote evidence of hepatic inflammation. The terms acute, subacute, and chronic are also ambiguous. Chronicity should refer to continuing or recurrent disease. Activity should refer to evidence of the presence of perpetuation of liver cell injury; this is most readily identified by serum transaminase elevations and by the degree of hepatocellular necrosis on biopsy.

Known therapeutic agents for liver damage serve to improve the function of liver tissue which has survived the initial cause of damage, however, the present goal is not simply to improve the condition of damaged tissue, but regeneration of liver cells. A known example of such an agent is hepatocyte growth factor (HGF), which stimulates the proliferation of cultured hepatocytes, and has an effect during cell division of hepatocytes, particularly during the GI phase (DNA synthesis preinterphase) of the cell cycle. It is recognized as the major factor causing migration of hepatocytes to the S phase (DNA synthesis phase). This factor was expected to induce liver regeneration in the clinic as well. However, no improvement in the condition of patients with acute liver failure was observed despite high concentrations of HGF in the peripheral blood. Thus an awareness is growing of the necessity of participation of a factor other than HGF for liver regeneration, but so far no such factor capable of inducing liver regeneration in vivo to any significant degree has been discovered.

### SUMMARY OF THE INVENTION

The invention provides a novel serine protease inhibitor, human regeneration-associated serpin-1 (RASP-1) polypeptide, and nucleic acids that encode RASP-1. RASP-1 is expressed at basal levels in the liver and is expressed at increased levels during the process of regeneration of the liver.

The invention also provides methods for detecting alterations in RASP-1 gene expression, which can be used in the diagnosis or prognosis of liver associated disorders. Methods for treating disorders of the liver, including treating partial hepatectomy, in which the expression and/or activity of an RASP-1 is modulated, are also included in the invention.

In yet another embodiment, the invention provides a non-coding regulatory region of the human RASP-1 gene. Specifically, the invention includes an isolated nucleic acid construct, comprising a non-coding regulatory sequence isolated upstream, downstream or within introns of a human RASP-1 gene and a heterologous nucleic acid sequence operably linked to the non-coding sequence wherein expression of the heterologous sequence is regulated by the non-coding sequence. Preferably the non-coding regulatory region is the RASP-1 promoter which provides tissue specific expression in the liver.

### BRIEF DESCRIPTION OF THE DRAWINGS

FIG. 1 shows a partial amino acid sequence of human RASP-1 as aligned with rat RASP-1. The 5′ amino acid sequence of human RASP-1 includes amino residues 1-53 (SEQ ID NO:2) and SEQ ID NO:3 includes 70 amino residues at the 3′ end of human RASP-1.

FIG. 2 shows an immunoblot of human serum contacted with pre-immune serum (lane 3), immune-serum (lane 5), pre-immune, albumin blocked serum (lane 7) or albumin blocked serum (lane 9). Molecular weight markers are indicated in lane 1.

### DETAILED DESCRIPTION OF THE INVENTION

The invention provides a novel serine protease inhibitor, regeneration-associated serpin-1 (RASP-1), and nucleic acids that encode RASP-1. The polypeptide is involved in regeneration of the liver, and therefore is useful in the diagnosis, prognosis and treatment of liver-associated disorders.

RASP-1 is expressed in the normal liver and is up-regulated in the regenerating liver. Accordingly, RASP-1 polypeptides, and nucleic acids that encode them, can be used in methods for treating and diagnosing conditions affecting the liver, e.g., regeneration of damaged liver tissue or cancer. Monoclonal and polyclonal antibodies can be produced using standard immunization and screening methods well known in the art. These antibodies can be easily detectably labeled and used histologically to identify tissues which contain RASP-1. RASP-1 can also be used in methods for maintaining cultured cells or tissues, such as hepatocytes cells or tissues, prior to transplantation, for example. In addition, RASP-1 can be used to promote hepatocyte growth in vitro, in order to, for example, facilitate production of growth factors, such as hepatocyte growth factor (HGF), that are produced by them. Methods employing RASP-1 polypeptide and nucleic acids are described in further detail below.

The invention provides substantially pure human RASP-1 polypeptide. The term “substantially pure” is used herein to describe a molecule, such as a polypeptide (e.g, an RASP-1 polypeptide, or a fragment thereof) that is substantially free of other proteins, lipids, carbohydrates, nucleic acids, and other biological materials with which it is naturally associated. For example, a substantially pure molecule, such as a polypeptide, can be at least 60%, by dry weight, the molecule of interest. One skilled in the art can purify RASP-1 polypeptides using standard protein purification methods and the purity of the polypeptides can be determined using standard methods including, e.g., polyacrylamide gel electrophoresis (e.g., SDS-PAGE), column chromatography (e.g., high performance liquid chromatography (HPLC)), and amino-terminal amino acid sequence analysis.

The RASP-1 polypeptides of the invention can be derived from a mammal, such as a human or a mouse. Also included in the invention are polypeptides having sequences that are “substantially identical” to the sequence of an RASP-1 polypeptide. A “substantially identical” amino acid sequence is a sequence that differs from a reference sequence only by conservative amino acid substitutions, for example, substitutions of one amino acid for another of the same class (e.g., substitution of one hydrophobic amino acid, such as isoleucine, valine, leucine, or methionine, for another, or substitution of one polar amino acid for another, such as substitution of arginine for lysine, glutamic acid for aspartic acid, or glutamine for asparagine), or by one or more non-conservative substitutions, deletions, or insertions, provided that the polypeptide retains at least one RASP-1-specific activity or an RASP-1-specific epitope. For example, one or more amino acids can be deleted from an RASP-1 polypeptide, resulting in modification of the structure of the polypeptide, without significantly altering its biological activity. For example, amino- or carboxyl-terminal amino acids that are not required for RASP-1 biological activity, can be removed. Such modifications can result in the development of smaller active RASP-1 polypeptides.

Other RASP-1 polypeptides included in the invention are polypeptides having amino acid sequences that are at least 50% identical to the amino acid sequence of an RASP-1 polypeptide. The length of comparison in determining amino acid sequence homology can be, for example, at least 15 amino acids, for example, at least 20, 25, or 35 amino acids. Homology can be measured using standard sequence analysis software (e.g., Sequence Analysis Software Package of the Genetics Computer Group, University of Wisconsin Biotechnology Center, 1710 University Avenue, Madison, Wis. 53705; also see Ausubel, et al., supra).

The invention also includes fragments of RASP-1 polypeptides that retain at least one RASP-1-specific activity or epitope. For example, an RASP-1 polypeptide fragment containing, e.g., at least 8-10 amino acids can be used as an immunogen in the production of RASP-1-specific antibodies. In addition to their use as peptide immunogens, the above-described RASP-1 fragments can be used in immunoassays, such as ELISAs, to detect the presence of RASP-1-specific antibodies in samples.

The RASP-1 polypeptides of the invention can be obtained using any of several standard methods. For example, RASP-1 polypeptides can be produced in a standard recombinant expression system (see below), chemically synthesized (this approach may be limited to small RASP-1 peptide fragments), or purified from tissues in which they are naturally expressed (see e.g., Ausubel, et al., supra).

The invention also provides isolated nucleic acid molecules that encode the RASP-1 polypeptide described above, as well as fragments thereof. These nucleic acids can contain naturally occurring nucleotide sequences, or sequences that differ from those of the naturally occurring nucleic acids that encode RASP-1, but encode the same amino acids, due to the degeneracy of the genetic code. The nucleic acids of the invention can contain DNA or RNA nucleotides, or combinations or modifications thereof.

The term “isolated nucleic acid” refers to a nucleic acid, e.g., a DNA or RNA molecule, that is not immediately contiguous with the 5′ and 3′ flanking sequences with which it normally is immediately contiguous when present in the naturally occurring genome of the organism from which it is derived. The term thus describes, for example, a nucleic acid that is incorporated into a vector, such as a plasmid or viral vector; a nucleic acid that is incorporated into the genome of a heterologous cell (or the genome of a homologous cell, but at a site different from that at which it naturally occurs); and a nucleic acid that exists as a separate molecule, e.g., a DNA fragment produced by PCR amplification or restriction enzyme digestion, or an RNA molecule produced by in vitro transcription. The term also describes a recombinant nucleic acid that forms part of a hybrid gene encoding additional polypeptide sequences that can be used, for example, in the production of a fusion protein.

The nucleic acid molecules of the invention can be used as templates in standard methods for production of RASP-1 gene products (e.g., RASP-1 RNAs and RASP-1 polypeptides. In addition, the nucleic acid molecules that encode RASP-1 polypeptide (and fragments thereof) and related nucleic acids, such as (1) nucleic acids containing sequences that are complementary to, or that hybridize to, nucleic acids encoding RASP-1 polypeptides, or fragments thereof (e.g., fragments containing at least 12, 15, 20, or 25 nucleotides); and (2) nucleic acids containing sequences that hybridize to sequences that are complementary to nucleic acids encoding RASP-1 polypeptides, or fragments thereof (e.g., fragments containing at least 12, 15, 20, or 25 nucleotides); can be used in methods focused on their hybridization properties. For example, as is described in further detail below, such nucleic acid molecules can be used in the following methods: PCR methods for synthesizing RASP-1 nucleic acids, methods for detecting the presence of an RASP-1 nucleic acid in a sample, screening methods for identifying nucleic acids encoding new RASP-1 family members, and therapeutic methods.

The invention also includes methods for identifying nucleic acid molecules that encode members of the RASP-1 polypeptide family in addition to RASP-1 (e.g., other serpins). In these methods, a sample, e.g., a nucleic acid library, such as a cDNA library, that contains a nucleic acid encoding an RASP-1 polypeptide is screened with an RASP-1-specific probe, e.g, an RASP-1-specific nucleic acid probe. RASP-1-specific nucleic acid probes are nucleic acid molecules (e.g., molecules containing DNA or RNA nucleotides, or combinations or modifications thereof) that specifically hybridize to nucleic acids encoding RASP-1 polypeptides, or to complementary sequences thereof. The term “RASP-1-specific probe,” in the context of this method of invention, refers to probes that bind to nucleic acids encoding RASP-1 polypeptides, or to complementary sequences thereof, to a detectably greater extent than to nucleic acids encoding other RASP-1 family members, or to complementary sequences thereof.

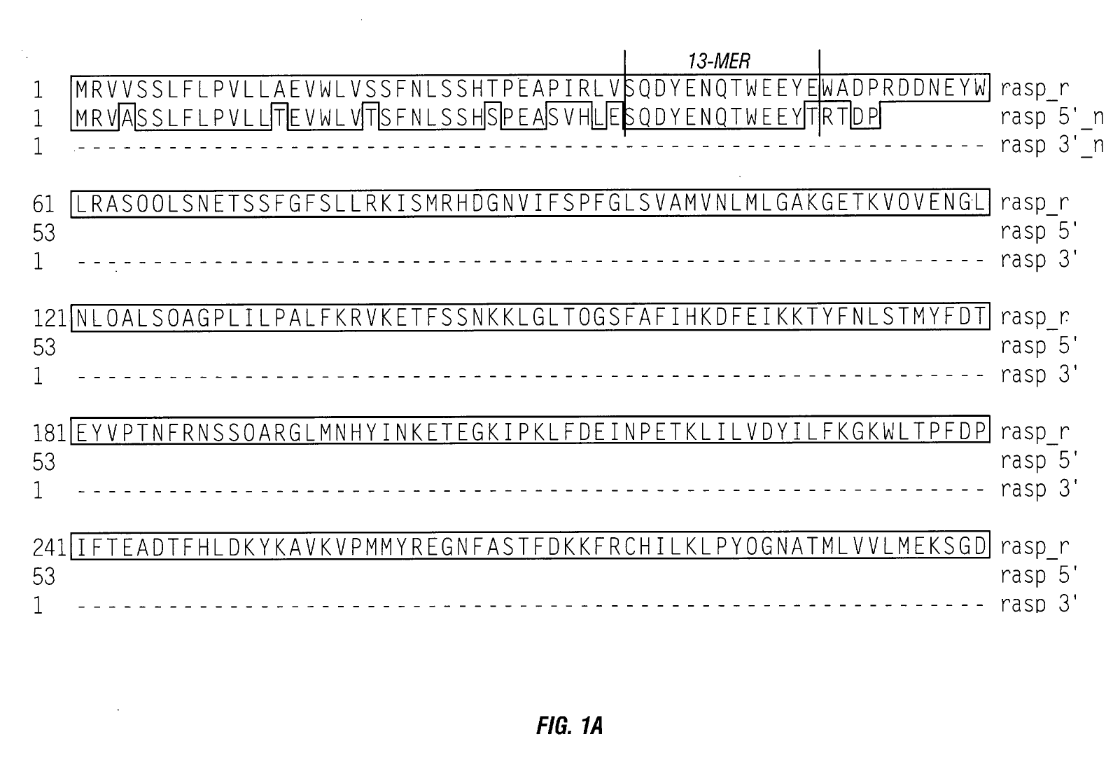

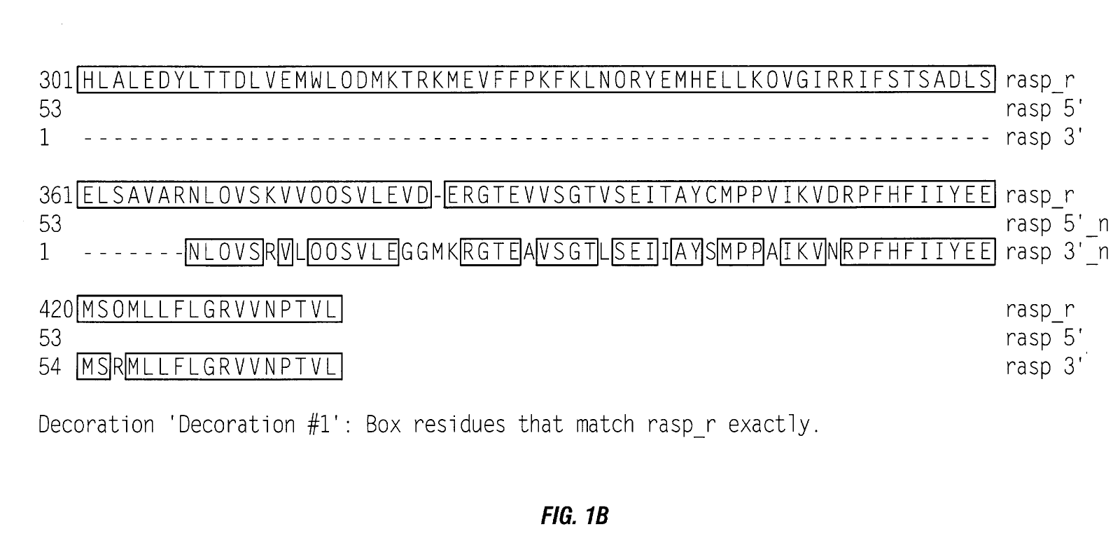

The invention facilitates production of RASP-1-specific nucleic acid probes. Methods for obtaining such probes can be designed based on the amino acid sequence shown in FIG. 1. The probes, which can contain at least 12, e.g., at least 15, 25, 35, 50, 100, or 150 nucleotides, can be produced using any of several standard methods (see e.g., Ausubel, et al., supra). For example, preferably, the probes are generated using PCR amplification methods. Once RASP-1-specific amino acid sequences are selected as templates against which primer sequences are to be designed, the primers can be synthesized using, e.g., standard chemical methods. As is described above, due to the degeneracy of the genetic code, such primers should be designed to include appropriate degenerate sequences, as can easily be determined by one skilled in the art.

In addition to RASP-1-specific nucleic acid probes, RASP-1-specific polypeptide probes, such as RASP-1-specific antibodies, can be used to screen samples, e.g., expression libraries, for nucleic acids encoding novel RASP-1 polypeptides, or portions thereof. For example, an antibody that specifically binds to an RASP-1-specific peptide can be used in this method. Methods for carrying out such screening are well known in the art (see e.g., Ausubel, supra).

The sequences of a pair of nucleic acid molecules (or two regions within a single nucleic acid molecule) are said to be “complementary” to each other if base pairing interactions can occur between each nucleotide of one of the members of the pair and each nucleotide of the other member of the pair. A pair of nucleic acid molecules (or two regions within a single nucleic acid molecule) are said to “hybridize” to each other if they form a duplex by base pairing interactions between them. As is known in the art, hybridization between nucleic acid pairs does not require complete complementarity between the hybridizing regions, but only that there is a sufficient level of base pairing to maintain the duplex under the hybridization conditions used.

Hybridization reactions are typically carried out under low to moderate stringency conditions, in which specific and some non-specific interactions can occur. After hybridization, washing can be carried out under moderate or high stringency conditions to eliminate non-specific binding. As is known in the art, optimal washing conditions can be determined empirically, e.g., by gradually increasing the stringency. Condition parameters that can be changed to affect stringency include, e.g., temperature and salt concentration. In general, the lower the salt concentration and the higher the temperature, the higher the stringency. For example, washing can be initiated at a low temperature (e.g., room temperature) using a solution containing an equivalent or lower salt concentration as the hybridization solution. Subsequent washing can be carried out using progressively warmer solutions having the same salt solution. Alternatively, the salt concentration can be lowered and the temperature maintained in the washing step, or the salt concentration can be lowered and the temperature increased. Additional parameters can be altered to affect stringency, including, e.g., the use of a destabilizing agent, such as formamide.

In nucleic acid hybridization reactions, the conditions used to achieve a particular level of stringency will vary, depending on the nature of the nucleic acids being hybridized. For example, the length, degree of complementarity, nucleotide sequence composition (e.g, GC v. AT content), and nucleic acid type (e.g., RNA v. DNA) of the hybridizing regions of the nucleic acids can be considered in selecting hybridization conditions. An additional consideration is whether one of the nucleic acids is immobilized, for example, on a filter.

An example of progressively higher stringency conditions is as follows: 2× SSC/0.1% SDS at about room temperature (hybridization conditions); 0.2× SSC/0.1% SDS at about room temperature (low stringency conditions); 0.2× SSC/0.1% SDS at about 42° C. (moderate stringency conditions); and 0.1× SSC at about 68° C. (high stringency conditions). Washing can be carried out using only one of these conditions, e g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

The nucleic acid molecules of the invention can be obtained by any of several standard methods. For example, the molecules can be produced using standard recombinant, enzymatic (e.g., PCR or reverse transcription (RT)/PCR methods), and chemical (e.g., phosphoramidite-based synthesis) methods. In addition, they can be isolated from samples, such as nucleic acid libraries and tissue samples (e.g., liver), using standard hybridization methods. For example, as described above, using standard methods, genomic or cDNA libraries can be hybridized with nucleic acid probes corresponding to RASP-1 nucleic acid sequences to detect the presence of a homologous nucleotide sequence in the library (see e.g., Ausubel, et al., supra). These methods are described in more detail above. Also as described above, nucleic acids encoding polypeptides containing at least one RASP-1 epitope, such as an RASP-1-specific epitope, can also be identified by screening a cDNA expression library, such as a library contained in lambda gtl 1, with an RASP-1-specific antibody as a probe. Such antibodies can be either polyclonal or monoclonal and are produced using standard methods (see, e.g., Harlow and Lane, Antibodies: A Laboratory Manual, Cold Spring Harbor Laboratory Press, Cold Spring Harbor, N.Y., 1988).

The RASP-1 nucleic acid molecules can be inserted into vectors, such as plasmid or viral vectors, that facilitate (1) expression of the inserted nucleic acid molecule and/or (2) amplification of the insert. As is well known in the art, such vectors can contain, e.g., promoter sequences, which facilitate transcription of the inserted nucleic acid in the cell, origins of replication, and genes, such as a neomycin-resistance gene, which encodes a selectable marker that imparts G418 resistance to cells in which it is expressed, and thus permits phenotypic selection of transformed cells.

Vectors suitable for use in the present invention include, e.g., T7-based expression vectors for use in bacteria (see e.g., Rosenberg, et al., Gene, 56:125, 1987), the pMSXND expression vector for use in mammalian cells (Lee and Nathans, J. Biol. Chem., 263:3521, 1988), and baculovirus-derived vectors for use in insect cells. The nucleic acids in such vectors are operably linked to a promoter, which is selected based on, e.g., the cell type in which expression is sought. For example, a T7 promoter can be used in bacteria, a polyhedrin promoter can be used in insect cells, and a cytomegalovirus or metallothionein promoter can be used in mammalian cells. Also, in the case of higher eukaryotes, tissue-specific promoters are available. (See e.g., Ausubel, et al., supra, for additional appropriate vectors and promoters that can be used in the invention; also see Pouwels, et al., Cloning Vectors: A Laboratory Manual, 1985, Supp. 1987). Viral vectors that can be used in the invention include, for example, retroviral, adenoviral, adeno-associated viral, herpes virus, simian virus 40 (SV40), and bovine papilloma virus vectors (see e.g, Gluzman ed., Eukaryotic Viral Vectors, Cold Spring Harbor Laboratory Press, Cold Spring Harbor, N.Y., 1982), and are discussed further below.

Cells into which RASP-1 nucleic acids can be introduced, in order to, for example, produce RASP-1 polypeptides using, e.g., the vectors described above, include prokaryotic cells (e.g., bacterial cells, such as E. coli cells) and eukaryotic cells (e.g., yeast cells, such as Saccharomyces cerevisiae cells; insect cells, such as Spodoptera frugiperda cells (e.g., Sf-9 cells); and mammalian cells, such as CHO, Cos-1, NIH-3T3, and JEG3 cells). Such cells are available from a number of different sources that are known to those skilled in the art, e.g., the American Type Culture Collection (ATCC), Rockville, Md. (also see, Ausubel, et al., supra). Cells into which the nucleic acids of the invention have been introduced, as well as their progeny, even if not identical to the parental cells, due to mutations, are included in the invention.

Methods for introducing the nucleic acids of the invention (e.g., nucleic acids inserted into the vectors described above) into cells, either transiently or stably, are well known in the art (see e.g., Ausubel, et al., supra). For example, in the case of prokaryotic cells, such as E. coli cells, competent cells, which are prepared from exponentially growing bacteria using a standard CaCl₂ (or MgCl₂ or RbCl) method, can be transformed using standard methods. Transformation of bacterial cells can also be performed using protoplast fusion methods. In the case of eukaryotic cells, transfection can be carried out using calcium phosphate precipitation or conventional mechanical procedures, such as microinjection and electroporation, can be used. Also, the nucleic acid (e.g., contained in a plasmid) can be packaged in a liposome using standard methods. In the case of viral vectors, appropriate infection methods, which are well known in the art, can be used (see e.g., Ausubel, et al., supra). In addition to being transfected with a nucleic acid encoding an RASP-1 polypeptide of the invention, eukaryotic cells, such as mammalian cells, can be co-transfected with a second nucleic acid encoding a selectable marker, such as a neomycin resistance gene or the herpes simplex virus thymidine kinase gene. As is mentioned above, such selectable markers can facilitate selection of transformed cells.

Isolation and purification of polypeptides produced in the systems described above can be carried out using conventional methods, appropriate for the particular system. For example, preparative chromatography and immunological separations employing antibodies, such as monoclonal or polyclonal antibodies, can be used.

Antibodies, such as monoclonal and polyclonal antibodies, that specifically bind to RASP-1 polypeptides (e.g., any or all of RASP-1) are also included in the invention. These antibodies can be made by using an RASP-1 polypeptide, or an RASP-1 polypeptide fragment that contains an RASP-1 epitope, as an immunogen in standard antibody production methods (see e.g., Kohler, et al., Nature, 256:495, 1975; Ausubel, et al., supra; Harlow and Lane, supra).

The term “antibody,” as used herein, refers to intact immunoglobulin molecules, as well as fragments of immunoglobulin molecules, such as Fab, Fab′, (Fab′)₂, Fv, and SCA fragments, that are capable of binding to an epitope of an RASP-1 polypeptide. These antibody fragments, which retain some ability to selectively bind to the antigen (e.g., an RASP-1 antigen) of the antibody from which they are derived, can be made using well known methods in the art (see, e.g., Harlow and Lane, supra), and are described further, as follows.

(1) An Fab fragment consists of a monovalent antigen-binding fragment of an antibody molecule, and can be produced by digestion of a whole antibody molecule with the enzyme papain, to yield a fragment consisting of an intact light chain and a portion of a heavy chain.

(2) An Fab′ fragment of an antibody molecule can be obtained by treating a whole antibody molecule with pepsin, followed by reduction, to yield a molecule consisting of an intact light chain and a portion of a heavy chain. Two Fab′ fragments are obtained per antibody molecule treated in this manner.

(3) An (Fab′)₂ fragment of an antibody can be obtained by treating a whole antibody molecule with the enzyme pepsin, without subsequent reduction. A (Fab′)₂ fragment is a dimer of two Fab′ fragments, held together by two disulfide bonds.

(4) An Fv fragment is defined as a genetically engineered fragment containing the variable region of a light chain and the variable region of a heavy chain expressed as two chains.

(5) A single chain antibody (“SCA”) is a genetically engineered single chain molecule containing the variable region of a light chain and the variable region of a heavy chain, linked by a suitable, flexible polypeptide linker.

As used in this invention, the term “epitope” refers to an antigenic determinant on an antigen, such as an RASP-1 polypeptide, to which the paratope of an antibody, such as an RASP-1-specific antibody, binds. Antigenic determinants usually consist of chemically active surface groupings of molecules, such as amino acids or sugar side chains, and can have specific three-dimensional structural characteristics, as well as specific charge characteristics.

As is mentioned above, antigens that can be used in producing RASP-1-specific antibodies include RASP-1 polypeptides or RASP-1 polypeptide fragments. The polypeptide or peptide used to immunize an animal can be obtained by standard recombinant, chemical synthetic, or purification methods. As is well known in the art, in order to increase immunogenicity, an antigen can be conjugated to a carrier protein. Commonly used carriers include keyhole limpet hemocyanin (KLH), thyroglobulin, bovine serum albumin (BSA), and tetanus toxoid. The coupled peptide is then used to immunize the animal (e.g., a mouse, a rat, or a rabbit). In addition to such carriers, well known adjuvants can be administered with the antigen to facilitate induction of a strong immune response.

RASP-1-specific polyclonal and monoclonal antibodies can be purified, for example, by binding to, and elution from, a matrix containing an RASP-1 polypeptide, e.g., the RASP-1 polypeptide (or fragment thereof) to which the antibodies were raised. Additional methods for antibody purification and concentration are well known in the art and can be practiced with the RASP-1-specific antibodies of the invention (see, for example, Coligan, et al., Unit 9, Current Protocols in Immunology, Wiley Interscience, 1994).

Anti-idiotype antibodies corresponding to RASP-1-specific antigens are also included in the invention, and can be produced using standard methods. These antibodies are raised to RASP-1-specific antibodies, and thus mimic RASP-1-specific epitopes.

The members of a pair of molecules (e.g., an antibody-antigen pair or a nucleic acid pair) are said to “specifically bind” to each other if they bind to each other with greater affinity than to other, non-specific molecules. For example, an antibody raised against an antigen to which it binds more efficiently than to a non-specific protein can be described as specifically binding to the antigen. (Similarly, a nucleic acid probe can be described as specifically binding to a nucleic acid target if it forms a specific duplex with the target by base pairing interactions (see above).)

As is discussed above, because of its tissue expression localization, RASP-1 is thought to play a role in the process of regeneration of the liver. Altered levels of RASP-1, such as increased levels, may thus be associated with cell proliferative disorders, such as cell proliferative disorders of the liver. The term “cell-proliferative disorder” is used herein to describe conditions that are characterized by abnormally excessive cell growth, including malignant, as well as non-malignant, cell growth. Conversely, conditions characterized by inadequate cell growth may be characterized by decreased expression of RASP-1. Accordingly, these conditions can be diagnosed and monitored by detecting the levels of RASP-1 in patient samples.

RASP-1-specific antibodies and nucleic acids can be used as probes in methods to detect the presence of an RASP-1 polypeptide (using an antibody) or nucleic acid (using a nucleic acid probe) in a sample, such as a biological fluid (e.g., plasma) or a tissue sample (e.g., liver, including hepatocytes and stromal cells). In these methods, an RASP-1-specific antibody or nucleic acid probe is contacted with a sample from a patient suspected of having an RASP-1-associated disorder, and specific binding of the antibody or nucleic acid probe to the sample detected. The level of RASP-1 polypeptide or nucleic acid present in the suspect sample can be compared with the level in a control sample, e.g., an equivalent sample from an unaffected individual, to determine whether the patient has an RASP-1-associated cell proliferative disorder. RASP-1 polypeptides, or fragments thereof, can also be used as probes in diagnostic methods, for example, to detect the presence of RASP-1-specific antibodies in samples.

The RASP-1-specific nucleic acid probes can be labeled with a compound that facilitates detection of binding to the RASP-1 nucleic acid in the sample. For example, the probe can contain biotinylated nucleotides, to which detectably labeled avidin conjugates (e.g. horse-radish peroxidase-conjugated avidin) can bind. Radiolabeled nucleic acid probes can also be used. There are many different labels and methods of labeling known to those of ordinary skill in the art. Examples of the types of labels which can be used in the present invention include enzymes, radioisotopes, fluorescent compounds, colloidal metals, chemiluminescent compounds, phosphorescent compounds, and bioluminescent compounds. Those of ordinary skill in the art will know of other suitable labels for binding to the antibody or probes, or will be able to ascertain such, using routine experimentation.

Another technique which may also result in greater sensitivity consists of coupling the antibodies to low molecular weight haptens. These haptens can then be specifically detected by means of a second reaction. For example, it is common to use such haptens as biotin, which reacts with avidin, or dinitrophenyl, puridoxal, and fluorescein, which can react with specific antihapten antibodies.

These probes can be used in nucleic acid hybridization assays to detect altered levels of RASP-1 in a sample. For example, in situ hybridization, RNAse protection, and Northern Blot methods can be used. Other standard nucleic acid detection methods that can be used in the invention are known to those of skill in the art (see e.g., Ausubel, et al., supra). In addition, when the diagnostic molecule is a nucleic acid, it can be amplified prior to binding with an RASP-1-specific probe. Preferably, PCR is used, but other nucleic acid amplification methods, such as the ligase chain reaction (LCR), ligated activated transcription (LAT), and nucleic acid sequence-based amplification (NASBA) methods can be used.

Use of RASP-1-specific antibodies in diagnostic and prognostic methods is described further, as follows. The antibodies of the invention can be used in vitro or in vivo for immunodiagnosis. The antibodies are suited for use in, for example, immuno-assays in which they are in liquid phase or bound to a solid phase carrier (e.g., a glass, polystyrene, polypropylene, polyethylene, dextran, nylon, amylase, natural and modified cellulose, polyacrylamide, agarose, or magnetite carrier). The antibodies used in such immunoassays can be detectably labeled (e.g., with an enzyme, a radioisotope, a fluorescent compound, a colloidal metal, a chemiluminescent compound, a phosphorescent compound, or a bioluminescent compound) using any of several standard methods that are well known in the art. Examples of immunoassays in which the antibodies of the invention can be used include, e.g., competitive and non-competitive immunoassays, which are carried out using either direct or indirect formats. Examples of such immunoassays include radioimmunoassays (RIA) and sandwich assays (e.g., enzvme-linked immunosorbent assays (ELISAs)). Detection of antigens using the antibodies of the invention can be done using imrnunoassays that are run in either forward, reverse, or simultaneous modes, including immunohistochemical assays on physiological samples. Other immunoassay formats are well known in the art, and can be used in the invention (see e.g., Coligan, et al., supra).

In addition to the in vitro methods described above, RASP-1-specific monoclonal antibodies can be used in methods for in vivo detection of an antigen, such as an RASP-1. In these methods, a detectably labeled antibody is administered to a patient in a dose that is determined to be diagnostically effective by one skilled in the art. The term “diagnostically effective” is used herein to describe the amount of detectably labeled monoclonal antibody that is administered in a sufficient quantity to enable detection of the site having the antigen for which the monoclonal antibody is specific. As would be apparent to one skilled in the art, the concentration of detectably labeled monoclonal antibody that is administered should be sufficient so that the binding of the antibody to the cells containing the polypeptide is detectable, compared to background. Further, it is desirable that the detectably labeled monoclonal antibody is rapidly cleared from the circulatory system, to give the optimal target-to-background signal ratio.

The dosage of detectably labeled monoclonal antibodies for in vivo diagnosis will vary, depending on such factors as the age and weight of the individual, as well as the extent of the disease. The dosages can also vary depending on factors such as whether multiple administrations are intended, antigenic burden, and other factors known to those of skill in the art.

In addition to initial diagnosis, the RASP-1 polypeptides, nucleic acids, and RASP-1-specific antibodies described above can be used prognostically using in vitro or in vivo methods for monitoring the progress of a condition associated with RASP-1 expression. For example, they can be used in methods to monitor the course of amelioration of an RASP-1-associated disease, for example, after treatment has begun. In these methods, changes in the levels of an RASP-1-specific marker (e.g., an RASP-1 polypeptide, an RASP-1 nucleic acid, or an RASP-1-specific antibody) are detected, either in a sample from a patient or using the in vivo methods described above. For example, it may be desirable to monitor hepatocyte proliferation during the regeneration process by monitoring RASP-1 expression.

The invention also provides methods for treating conditions associated with altered expression of RASP-1 polypeptides, for example, cell proliferative disorders (e.g., cell proliferative disorders of the liver). The method of the invention can be used with subjects having or at risk of having (e.g., familial disease or alcoholism) such a disorder. Treatment of a RASP-1-associated cell proliferative disorder can be carried out, for example, by modulating RASP-1 gene expression or RASP-1 activity in a cell. The term “modulate” includes, for example, suppressing expression of an RASP-1 when it is over-expressed, and augmenting expression of an RASP-1 when it is under-expressed. In cases where a cell-proliferative disorder is associated with over-expression of an RASP-1, nucleic acids that interfere with RASP-1 expression, at transcriptional or translational levels, can be used to treat the disorder. This approach employs, for example, antisense nucleic acids (i.e., nucleic acids that are complementary to, or capable of hybridizing with, a target nucleic acid, e.g., a nucleic acid encoding an RASP-1 polypeptide), ribozymes, or triplex agents. The antisense and triplex approaches function by masking the nucleic acid, while the ribozyme strategy functions by cleaving the nucleic acid. In addition, antibodies that bind to RASP-1 polypeptides can be used in methods to block the activity of an RASP-1.

The use of antisense methods to inhibit the in vitro translation of genes is well known in the art (see, e.g., Marcus-Sakura, Anal. Biochem., 172:289, 1988). Antisense nucleic acids are nucleic acid molecules (e.g., molecules containing DNA nucleotides, RNA nucleotides, or modifications (e.g., modification that increase the stability of the molecule, such as 2′-O-alkyl (e.g., methyl) substituted nucleotides) or combinations thereof) that are complementary to, or that hybridize to, at least a portion of a specific nucleic acid molecule, such as an RNA molecule (e.g., an mRNA molecule) (see, e.g., Weintraub, Scientific American, 26 :40, 1990). The antisense nucleic acids hybridize to corresponding nucleic acids, such as mRNAs, to form a double-stranded molecule, which interferes with translation of the mRNA, as the cell will not translate an double-stranded mRNA. Antisense nucleic acids used in the invention are typically at least 10-12 nucleotides in length, for example, at least 15, 20, 25, 50, 75, or 100 nucleotides in length. The antisense nucleic acid can also be as long as the target nucleic acid with which it is intended that it form an inhibitory duplex. As is described further below, the antisense nucleic acids can be introduced into cells as antisense oligonucleotides, or can be produced in a cell in which a nucleic acid encoding the antisense nucleic acid has been introduced by, for example, using gene therapy methods.

In addition to blocking mRNA translation, oligonucleotides, such as antisense oligonucleotides, can be used in methods to stall transcription, such as the triplex method.

In this method, an oligonucleotide winds around double-helical DNA in a sequence-specific manner, forming a three-stranded helix, which blocks transcription from the targeted gene. These triplex compounds can be designed to recognize a unique site on a chosen gene (Maher, et al., Antisense Res. and Dev.,1(3):227, 1991; Helene, Anticancer Drug Design. 6(6):569, 1991). Specifically targeted ribozymes can also be used in therapeutic methods directed at decreasing RASP-1 expression.

Introduction of RASP-1 antisense nucleic acids into cells affected by a proliferative disorder, for the purpose of gene therapy, can be achieved using a recombinant expression vector, such as a chimeric virus or a colloidal dispersion system, such as a targeted liposome. Those of skill in this art know or can easily ascertain the appropriate route and means for introduction of sense or antisense RASP-1 nucleic acids, without resort to undue experimentation.

Gene therapy methods can also be used to deliver genes encoding RASP-1. These methods can be carried out to treat conditions associated with insufficient RASP-1 expression. Thus, these methods can be used to promote liver tissue repair or replacement, for example, in conditions including hepatitis or cirrhosis. Further, the methods can be used to treat a partial hepatectomy in a subject. Therefore, after the hepatectomy is performed in the subject, the desirable gene is administered to the subject in an amount effective to stimulate liver regeneration. Recombinant RASP-1 can be administered directly by any conventional recombinant protein administration techniques or administered systemically. RASP-1 may also be targeted to specific cells or receptors by any of the methods known in the art to target nucleic formulations. The actual dosage of RASP-1 protein depends on a number of factors, including the size and health of an organism, however one of one of ordinary skill in the art can use the following teachings describing the methods and techniques for determining clinical dosages (Bert Spilker, Guide to Clinical Studies and Developing Protocols, Raven Press Books, Ltd., New York, 1984, pp. 7-13, 54-60; Bert Spilker, Guide to Clinical Trials, Raven Press, Ltd., New York, 1991, pp. 93-101; Charles Craig and Robert Stitzel, eds., Modern Pharmacology, 2d ed., Little, Brown and Co., Boston, 1986, pp. 127-33; Trevor Speight, ed., Avery's Drug Treatment: Principles and Practice of Clinical Pharmacology and Therapeutics, 3d ed., Williams and Wilkins, Baltimore, 1987, pp. 50-56;Ronald Tallarida. Robert Raffa and Paul McGonigle, Principles in General Pharmacology, Springer-Veriag, New York, 1988, pp. 18-20) to determine the appropriate dosage to use; but, generally, between 0.1 mg and 100 mg inclusive are administered per day to an adult in any pharnaceutically-acceptable carrier.

Additionally, one of ordinary skill in the art can use known culturable liver cells or other systems, such as that of the isolated perfused rat liver (IPRL) to determine sufficient amounts of RASP-1 to stimulate proliferation of hepatocytes and assess normal hepatocyte function. Using either permanent liver cultures or IPRL, rigorous long-term experimental studies are conducted. Monolayer cultures using hepatocyte cells are typically maintained for several months and a bio-artificial liver device prepared with these cells functions normally over a prolonged period (e.g., eight weeks), as determined by albumin production and glucose utilization. Furthermore, cartridges containing cultured hepatocyte cells reflect human liver metabolism as well as the use of isolated perfused livers from other species. Thus, the clinical effects of various amounts of RASP-1 are assessed in a particularly effective in vitro model when culturable hepatocyte cells or IPRL are used.

Due to the high levels of expression of RASP-1 in the liver, there are a variety of applications for RASP-1-specific polypeptides, nucleic acids, and antibodies related to treating disorders of this tissue. Such applications include disorders related to RASP-1 expression in the liver. Various developmental or acquired disorders can also be treated using RASP-1-related molecules. Liver conditions include, for example, viral infection (e.g., hepatitis B or C virus), autoimmunity, hepatitis (e.g., viral, drug-induced, toxic, ischemic), cirrhosis (e.g., alcoholic, postnecrotic, biliary, hemochromatosis), infiltrations (e.g., glycogen, fat, amyloid, lymphoma, granuloma), space-occupying lesions (e.g., hepatoma, abscess, cysts, gummas), functional disorders (Gilbert's syndrome, Crigler-Najjar syndrome, Dubin-Johnson and Rotor syndromes, cholestasis of pregnancy and benign recurrent cholestasis), hepatobiliary disorders such as extrahepatic biliary obstruction (stone, stricture or tumor), or cholangitis (septic, primary biliary cirrhosis), and vascular liver diseases such as chronic passive congestion, hepatic vein thrombosis, portal vein thrombosis, pylephlebitis, arteriovenous malformations, and venocclusive disease.

In yet another embodiment, the invention includes an isolated nucleic acid construct, comprising a non-coding regulatory sequence isolated at least upstream from a human RASP-1 gene and a heterologous nucleic acid sequence operably linked to the non-coding sequence wherein expression of the heterologous sequence is regulated by the non-coding sequence. The regulatory region of RASP-1 allows tissue-specific expression (i.e. liver) of a gene operatively linked thereto.

The RASP-1 gene regulatory sequence which may include a promoter, enhancer and/or silencer, is located in the non-coding region of the gene and exhibits strong expression in liver tissue. 5′ non-coding sequence and possibly 3′ non-coding sequence isolated upstream and downstream, respectively, from the coding sequence, and/or intron sequences can be isolated from the RASP-1 nucleic acids provided in the present invention. The transcription initiation sequences will include transcriptional control regions such as TATAA and CAAT box sequences as well as sequences which regulate the tissue specificity of the transcribed product. In the nucleic acid construct of the invention, the ATG start codon is typically provided by the nucleic acid sequence expressing the product of interest.

One may identify a convenient restriction site in the 5′ or 3′-untranslated region of the gene and in the 5′ region of the nucleic acid sequence expressing the product of interest and employ an adapter which will join the two sequences. Alternatively, one may introduce a polylinker immediately downstream from the RASP-1 noncoding region for insertion of the nucleic acid sequence expressing the product of interest.

Placing a nucleic acid sequence expressing a product of interest under the regulatory control of a promoter or a regulatory element means positioning the sequence such that expression is controlled by the promoter or regulatory element. In general, promoters are positioned upstream of the genes that they control. Thus, in the construction of promoter/gene combinations, the promoter is preferably positioned upstream of the gene and at a distance from the transcription start site that approximates the distance between the promoter and the gene it controls in its natural setting. As is known in the art, some variation in this distance can be tolerated without loss of promoter function. Similarly, the preferred positioning of a regulatory element with respect to a gene placed under its control reflects its natural position relative to the structural gene it naturally regulates. Again, as is known in the art, some variation in this distance can be accommodated. The noncoding sequences which are used in the invention construct are not more than about 6-8 kbp in length.

Promoter function during expression of a gene under its regulatory control can be tested at the transcriptional stage using DNA/RNA and RNA/RNA hybridization assays (in situ hybridization) and at the translational stage using specific functional assays for the protein synthesized (for example, by enzymatic activity or by immunoassay of the protein).

In another embodiment, the invention provides a method of stimulating hepatocyte cell growth comprising contacting the hepatocyte with a sufficient amount of RASP-1 to stimulate proliferation of the hepatocyte. The method of the invention includes both in vitro and in vivo applications.

In vitro liver tissue culture can be used to support or replace the natural liver, by direct implantation or as part of an extracorporeal liver device. In addition, such liver tissue cultures can serve as models for testing toxicity of drugs or other compounds, as well as screening for agents useful for stimulating liver tissue, including pharmacological agents.

In situations where a patient's natural liver is damaged or diseased, it may be desirable to leave intact or to partially remove, but still require support from implanted artificial liver tissue or liver tissue transplanted from a donor. Pharmaceutical compositions comprising a Rasp-1 gene product can be used to enhance the growth of the patient's natural liver tissue, as well as the implanted or transplanted liver tissue.

It is to be understood that, while the invention has been described with reference to the above detailed description, the foregoing description is intended to illustrate, but not to limit, the scope of the invention. Other aspects, advantages, and modifications of the invention are within the scope of the following claims. All publications, patent applications, patents, and other references mentioned herein are incorporated by reference in their entirety.

The following examples are intended to illustrate, but not to limit, the invention. While the procedures described in the examples are typical of those that can be used to carry out certain aspects of the invention, other procedures known to those skilled in the art can also be used. The following materials and methods were used in carrying out the experiments described in the examples.

### EXAMPLES

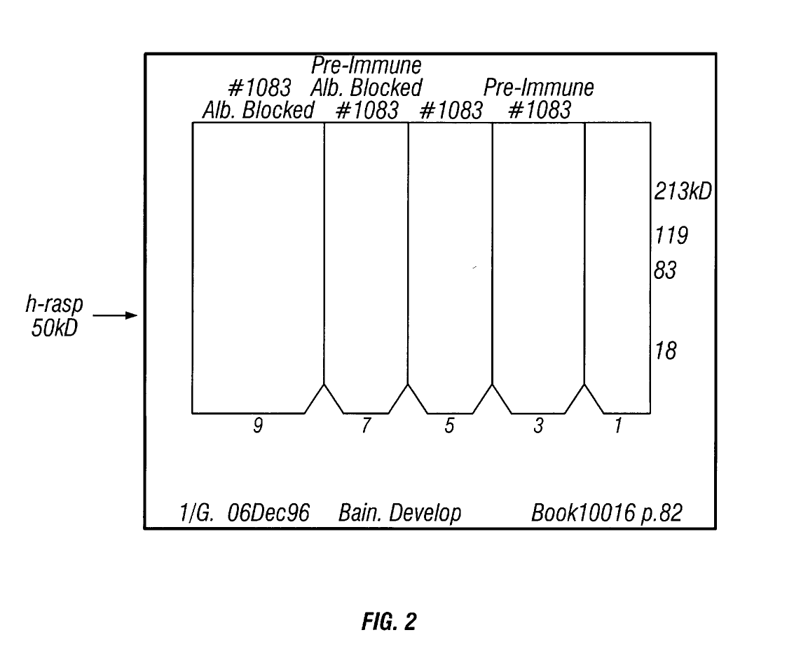

A search against the dBEST data base revealed a human EST fragment with significant homology to rat RASP-1. A peptide from the deduced amino acid sequence of human RASP-1 [SQDYENQIWEEYT] (SEQ ID) NO:1) was synthesized and used to immunize rabbits (immunization and serum collection performed by Genosys, Woodland, Tex.). For Western blot analysis, 0.2 microliters of human serum was electrophoresed on a 7.5% SDS-polyacrylamide gel, transferred to nitrocellulose membrane and blotted with either preimmune (FIG. 2, lane 3) or anti-human RASP-1 serum (FIG. 2, lane 5). The results show that the anti-human RASP-1 serum specifically reacts with an approximately 50 kD protein which is not detectable in the control lane.

Using the partial 5′ and 3′ sequences provided herein, the full length human RASP-1 cDNA and its homologues can be obtained from any standard human cDNA library. In particular for obtaining cDNA, tissues and cells in which RASP-1 is expressed are optimal. Tissues which provide an optimal source of genetic material for RASP-1 and its homologues include liver, including adult, embryonic, fetal and regenerating liver. Specific cellular sources include especially human derived liver cells such as HepG2, Hep3B or PLC/PLF/5. For example, the isolated RASP-1 gene sequence supplied herein may be labled and used to screen a cDNA library constructed from mRNA obtained from the genetic material described above. The hybridization conditions used should be of a lower stringency when the cDNA library is initially screened with increasing stringency applied as potential positive clones are subsequently screened. Alternatively, the labeled fragment may be used to screen a human genomic Elibray under appropriate hybridization conditions. (See, Sambrook et al., Current Edition, Molecular Cloning, A Laboratory Manual, Cold Spring Harbor Press, N.Y.).

Further, the full length human RASP-1 cDNA can be isolated by utilizing the polymerase chain reaction (PCR) according to well known art methods and those supplied below. (See, Eds. Dieffenbach and Dveksler, Current Edition, PCR Primer: A Laboratory Manual, Cold Spring Harbor Press, N.Y.). For example, RNA can be isolated, using standard procedures, from an appropriate cell or tissue source. A reverse transcription reaction may be performed on the rRNA usain an olognucloetide primer specific fro the most 5′ end of the amplified fragment for the priming of first strand synthesis. The resulting RNA/DNA hybrid may then be “tailed” with guanines using a standard terminal transferase reaction, the hybrid may be digested with RNAase H, and second strand synthesis may then be primed with a poly-C primer. Thus, cDNA sequences upstream of the amplified fragment can be easily isolated.

Alternatively, a cDNA expression library can by constructed and screened utililizing DNA isolated from or cDNA synthesized from liver tissue known or suspected of expressing RASP-1. Gene products made can be screened using standard antibody screening techniques in conjunction with the anti-RASP-1 antibody described above. (See, Eds. Harlow and Lane, Current Edition, Antibodies: A Laboratory Manual, Cold Spring Harbor Press, N.Y. for screening techniques). Library clones detected via their reaction with a labled antibody such as anti-RASP-1 can be subsequently isolated, purified and sequenced according to well known methods.

DNA probe labeling. 25 ng of cDNA is synthesized from normal liver mRNA or regenerating liver mRNA or 0.5 μg of DNA fragment purified from agarose gel are ³²P labeled using the Primer-It II random niner labeling kit (Stratagene, La Jolla, Calif.).

RNA extraction and Northern hybridization analysis. Total RNA is extracted in guanidine thiocyanate solution (Chomczymski, P. et al., Anal. Biochem., 1987, 162: 156-159). The poly(A+) containing fraction is obtained by passing isolated total RNA through an oligo(dT)-cellulose column (Stratagene, La Jolla, Calif.). For Northern blots, 10 to 15 μg of total RiNA is fractionated on a vertical 1% agarose gel containing 6% w/v formaldehyde and transferred onto a nylon membrane (Hybond N, Amersham) by capillary in 20× SSC buffer followed by UV-crosslinking. Prehybridization and hybridization are carried out at 42° C. in a solution of 50% formamide, 4× SSPE buffer, 2× Denhardts' reagent and 0.2% sodium dodecyl sulfate (SDS) with yeast tRNA at a concentration of 0.8 mg/ml. After hybridization, blots are washed in 0.1% SDS and 0.2× SSC at 65° C. Before exposure, filters are stained in a solution containing 0.5M sodium acetate (pH 4.8) and 0.05% methylene blue to ensure equal loading and transfer of RNA.

Construction and screening of cDNA library. A cDNA library is constructed in λ-uniZAP (Stratagene, La Jolla, Calif.). Briefly, 5 μg of regenerating liver poly(A+) RNA is reverse transcribed by M-MuL Reverse Transcaptase and DNA polymerase I is used for synthesis of second strand cDNAs. The cDNA termini are blunted by Klenow fragment and EcoRI adaptors are ligated onto cDNAs. The cDNA fragments with the EcoRI site at the 5′ end and the Xhol site at the 3′ end are directionally ligated to predigested λ-uniZAP and packaged in Gigapack Gold λ packaging extracts (Stratagene, La Jolla, Calif.) The entire library is amplified by plating on E. coli strain XL1-Blue MRF′. A total of 1.5×10⁵ library phages are plated and quadruplicate nitrocellulose membrane lifts for each plate are differentially screened with cDNA probes made from normal and regenerating liver mRNAs by hybridizing two of the four lifts to a different probe, respectively. The primary positive plaques showing increased hybridization to the regenerating liver probe are selected for a second round of differential screening by polymerase chain reaction (PCR) (Thomas, M. G., et al., BioTechniques, 1994, 16: 229-231). The cDNA in the second round positive phages is excised out of the phage in a Bluescapt plasmid by following the phagemid excision procedure (Stratagene, La Jolla, Calif.).

Analysis of cDNA sequences and predicted amino acid sequence. Positive isolates are purified and subjected to double strand nucleotide sequencing with T7 and T3 primers flanking the inserts in Bluescript. Nucleotide sequences of 200-bp are obtained from each end of the inserts. These nucleotide sequences and some of the deduced putative in-frame amino acid sequences are searched by Blast program (NCBI) to ascertain their homology and uniqueness (Altschul, S. F., et al., J. Mol. Biol., 1990, 215: 403-410). Putative rasp 1 clones are chosen for further study.

1. A substantially pure human regeneration-associated serpin-1 (RASP-1) polypeptide.

2. The polypeptide of claim 1, wherein the polypeptide is characterized as:
a) having a molecular weight of about 50 kD by reducing SDS-PAGE; and
b) having serine protease inhibitory activity.

3. The polypeptide of claim 1, wherein the polypeptide contains an amino acid sequence as set forth in SEQ ID NO:2 or SEQ ID NO:3.

4. An isolated nucleic acid encoding the RASP-1 polypeptide of claim 1.

5. An isolated nucleic acid selected from the group consisting of:
a) a nucleic acid sequence of claim 4, where T can also be U;
b) a nucleic acid sequence that hybridizes to the complement of the nucleic acid sequence of claim 4; and
c) a fragment of a) or b) that comprises at least 15 nucleotides and hybridizes to a nucleotide sequence encoding SEQ ID NO:2 or SEQ ID NO:3.

6. A nucleic acid that hybridizes to the nucleic acid of claim 4.

7. A nucleic acid that hybridizes to the nucleic acid of claim 6.

8. The nucleic acid of claim 4, wherein the nucleic acid is mammalian.

9. The nucleic acid of claim 8, wherein the nucleic acid is human.

10. An expression vector containing the nucleic acid of claim 4.

11. The vector of claim 10, wherein the vector is a plasmid.

12. The vector of claim 10, wherein the vector is a virus.

13. A cell stably transformed with the vector of claim 10.

14. An antibody that binds to the RASP-1 polypeptide of claim 1.

15. The antibody of claim 14, wherein the antibody is monoclonal.

16. A method of detecting a cell proliferative disorder associated with expression of RASP-1 polypeptide, the method comprising:
a) contacting a specimen containing RASP-1 polypeptide or polynucleotide from a subject having or at risk of having the disorder with a reagent that detects RASP-1 polypeptide or polynucleotide;
b) detecting binding of the reagent to the specimen; and
c) comparing the level of expression of RASP-1 with the level of expression of RASP-1 in a control specimen.

17. The method of claim 16, wherein the cell is a hepatocyte.

18. The method of claim 16, wherein the reagent is an antibody.

19. The method of claim 16, wherein the reagent is a nucleic acid.

20. The method of claim 19, wherein the nucleic acid hybridizes to the nucleic acid of claim 4.

21. The method of claim 20, wherein the nucleic acid hybridizes to the complement of the nucleic acid of claim 4.

22. The method of claim 16, wherein the detecting is in vivo.

23. The method of claim 16, wherein the detecting in vitro.

24. The method of claim 16, wherein the reagent comprises a detectable label.

25. A method of treating a cell proliferative disorder associated with expression of RASP-1 polypeptide, the method comprising administering to a subject having or suspected of having the disorder a reagent that suppresses the activity of the RASP-1 polypeptide.

26. The method of claim 25, wherein the reagent is an anti-RASP-1 antibody.

27. The method of claim 25, wherein the reagent is a nucleic acid that hybridizes to the nucleic acid of claim 4.

28. The method of claim 25, wherein the cell is a hepatocyte.

29. The method of claim 25, wherein the reagent is introduced into the cell using a carrier.

30. The method of claim 29, wherein the carrier is a vector.

31. A method of identifying a nucleic acid encoding an RASP-1 polypeptide, the method comprising probing a sample containing a nucleic acid encoding an RASP-1 polypeptide with a RASP-1-specific nucleic acid probe.

32. A method of gene therapy comprising introducing into cells of a host subject, an expression vector comprising a nucleotide sequence encoding RASP-1, in operable linkage with a promoter.

33. The method of claim 32, wherein the expression vector is introduced into the subject's cells ex vivo and the cells are then reintroduced into the subject.

34. The method of claim 32, wherein the expression vector is an RNA virus.

35. The method of claim 34, wherein the RNA virus is a retrovirus.

36. The method of claim 32, wherein the subject is a human.

37. The method of claim 32, wherein the cell is a hepatocyte.

38. A diagnostic kit for the detection of a target cellular component indicative of a cell proliferative disorder in a subject having or at risk of having a liver associated disorder, comprising carrier means containing one or more containers comprising a first container containing a probe for detection of RASP-1 nucleic acid or polypeptide.

39. The kit of claim 38, wherein the target cellular component is a RASP-1 polypeptide.

40. The kit of claim 39, wherein the probe is an antibody.

41. The kit of claim 39, wherein the target cellular component is a nucleic acid sequence.

42. The kit of claim 41, wherein the probe is a polynucleotide hybridization probe.

43. An isolated nucleic acid construct, comprising:
a non-coding regulatory sequence isolated at least upstream from a human RASP-1 gene; and a heterologous nucleic acid sequence operably linked to the non-coding sequence, wherein expression of the heterologous sequence is regulated by the non-coding sequence.

44. The construct of claim 43, wherein the heterologous sequence expresses a biologically active protein.

45. The construct of claim 43, wherein the heterologous sequence encodes antisense RNA which antisense disrupts expression of an endogenous coding sequence.

46. The construct of claim 43, wherein the non-coding sequence is derived from the nucleotide sequence shown in FIG. 1.

47. The construct of claim 43, wherein the non-coding sequence comprises a transcriptional and translational initiation region.

48. The construct of claim 43, further comprising a transcriptional termination region functional in an animal cell.

49. The construct of claim 43, wherein the nucleic acid sequence encodes a therapeutic reagent.

50. A method of stimulating hepatocyte cell growth comprising contacting the hepatocyte with a sufficient amount of RASP-1 to stimulate proliferation of the hepatocyte.

51. The method of claim 50, wherein the stimulation is in vitro.

52. The method of claim 50, wherein the stimulation is in vivo.
